(19) United States
(12) Patent Application Publication
(10) Pub. No.: US 2023/0194496 A1
(43) Pub. Date: Jun. 22, 2023

(54) METHOD FOR DETERMINING A CARBON CONTENT OF A SAMPLE AND TOC ANALYZER

(71) Applicant: Endress+Hauser Conducta GmbH+Co. KG, Gerlingen (DE)

(72) Inventors: Ulrich Kathe, Ludwigsburg (DE); Maik Röder, Benningen (DE); Kay Steen, Leinfelden-Echterdingen (DE)

(21) Appl. No.: 18/145,146

(22) Filed: Dec. 22, 2022

(30) Foreign Application Priority Data
Dec. 22, 2021 (DE) 10 2021 134 321.6

(51) Int. Cl. G01N 33/18; G01N 33/00; B01D 53/00

(52) U.S. Cl. CPC G01N 33/1846; B01D 53/002; G01N 33/0016; B01D 2257/504

(57) ABSTRACT

A method for determining a carbon content of a sample in a TOC analyzer, includes the steps of: directing a carrier gas from an inlet through a high temperature furnace to an analysis unit; stopping the flow of the carrier gas through the high temperature furnace; injecting the sample into the high temperature furnace, which is used to vaporize and/or oxidize the sample at a high temperature to form water vapor and carbon dioxide gas; waiting until the sample injected into the high temperature furnace is vaporized; starting the flow of the carrier gas through the high temperature furnace and thereby transporting the carbon dioxide gas produced during vaporization and/or oxidation of the sample to an analysis unit; and determining the carbon content of the sample by means of the analysis unit on the basis of the carbon dioxide gas produced during the oxidation of the sample.

### CROSS-REFERENCE TO RELATED APPLICATION

The present application is related to and claims the priority benefit of German Patent Application No. 10 2021 134 321.6, filed on Dec. 22, 2021, the entire contents of which are incorporated herein by reference.

### TECHNICAL FIELD

The present disclosure relates to a method for determining a carbon content of a sample in a TOC analyzer and to a TOC analyzer.

### BACKGROUND

A TOC analyzer determines at least the TOC content, i.e., the “total organic carbon” content, in a sample. TOC analyzers sometimes additionally determine the TIC, i.e., the “total inorganic carbon” content, or the TC, i.e., the “total carbon” content. The carbon content plays, for example, a major role in the analysis of water for contaminations, e.g., in wastewater, drinking water, sea water, and surface bodies of water, as well as in process water or in water for pharmaceutical applications.

In liquid samples, the carbon contained therein is typically converted to carbon dioxide either in a wet-chemical manner or using UV or combustion methods. The sample is combusted in a high-temperature furnace at 670-1,200° C. In combustion methods (in particular at temperatures of < 1,000° C.), a catalyst is often used to ensure complete oxidation. In aqueous samples, therefore, in addition to carbon dioxide and other combustion gases, water vapor also arises, and is generally condensed after the combustion and separated from the carbon dioxide gas. Before the carbon dioxide gas is passed into the analysis unit, dusts, aerosols, and other gas constituents are sometimes removed from the carbon dioxide gas using filters and absorbers or adsorbers. A stream of a carrier gas transports the carbon dioxide gas to the analysis unit. Oxygen or mixtures of oxygen with nitrogen or (processed) compressed and ambient air are used as carrier gas, for example. The carbon content is often determined by means of a non-dispersive infrared (NDIR) sensor.

In the TOC measurement via the catalytic high-temperature method, an aliquot of the aqueous sample is metered into the hot reactor. The sample itself should be representative of the medium as a whole, and homogeneous. Because the total organic carbon (“TOC”) also contains particles in addition to the aqueous phase, the sample must be homogenized, i.e., comminuted and mixed, before the actual analysis. A relatively large volume is required for this purpose, from which only a precisely known, small representative volume is metered into the reactor. There, this is vaporized, and the organic ingredients of the sample oxidize to CO₂. The CO₂ is, as mentioned, conducted by carrier gas to the CO₂ detector, and the CO₂ concentration in the carrier gas is measured. The CO₂ signal appears as a peak, such as a bell curve, and must be integrated over time. The “peak integral” is in turn proportional to the TOC concentration in the starting sample, after taking into account the sample volume used.

A problem with the metering of the aqueous sample in the reactor heated to, for example, 680° C. is that, on the one hand, the sample must vaporize suddenly in order to obtain the desired peak shape. On the other hand, relatively large sample quantities must be used in order to be able to measure in the trace range. If too much sample is metered into the reactor in a short time, it cannot vaporize suddenly. Depending upon the sample volume, it is then still in liquid form in the reactor for some time before it is completely vaporized. This widens and deforms the CO₂ curve, which can lead to measurement errors. In addition, it is technically very complicated to hold constant the flow rate of the carrier gas arriving at the CO₂ detector. If this effort is not put forth, measurement errors result.

In DE 199 31 801, the carrier gas speed, in addition to the CO₂ signal, is detected for the evaluation. Both signals are multiplied and integrated with one another. This compensates for the error due to inconstant carrier gas flow rates. An idealization of the curve shape does not take place.

In WO2019/032574, before the sample metering, the carrier gas flow is diverted around the reactor by switching a 3/2-way valve upstream and a further 3/2-way valve downstream of the reactor in a bypass. The sample is then slowly metered into the reactor. If the sample is thereafter completely vaporized in the reactor, and the catalyst is back to operating temperature, the carrier gas is passed back through the reactor to the CO₂ sensor. A great technical effort is required in order for the flow of the carrier gas to be kept constant. In addition, two valves are necessary.

In DE 11 2018 007 859 T, the curved shape of the CO₂ peaks after the sample has been introduced into the reactor is modified by adjustment of the carrier gas speed such that a Gaussian shape is obtained. This requires a high technical effort.

### SUMMARY

The object of the present disclosure is to provide a simple but reproducible solution in order to meter and vaporize larger amounts of sample into the reactor in TOC analyzers.

The object is achieved by a method for determining a carbon content of a sample in a TOC analyzer, comprising the steps of: directing a carrier gas from an inlet through a high temperature furnace to an analysis unit; stopping the flow of the carrier gas through the high temperature furnace; injecting the sample into the high temperature furnace, which is used to vaporize and/or oxidize the sample at a high temperature to form water vapor and carbon dioxide gas; waiting until the sample injected into the high temperature furnace is vaporized; starting the flow of the carrier gas through the high temperature furnace and thereby transporting the carbon dioxide gas produced during the vaporization and/or oxidation of the sample to an analysis unit; and determining the carbon content of the sample by means of the analysis unit on the basis of the carbon dioxide gas produced during the oxidation of the sample.

One embodiment provides for the injection to be performed in a pulse-like manner.

One embodiment provides that the determination of the carbon content be performed cyclically.

One embodiment provides that the flow, such as the mass flow, of the carrier gas through the analysis unit be measured by means of a flow meter.

One embodiment provides that the measured flow be multiplied by the carbon content of the sample, wherein this product is integrated over time, and the TOC concentration of the sample is determined from the integral.

The object is further achieved by a TOC analyzer for determining a carbon content of a sample, comprising an inlet for a carrier gas, wherein the inlet leads to a high temperature furnace via a shut-off device, wherein the carrier gas for is used for transporting a carbon dioxide gas produced in the high-temperature furnace during an oxidation of the sample to an analysis unit; the shut-off device for stopping and starting the flow of the carrier gas through the high-temperature furnace; an injection unit for the injecting the sample into the high-temperature furnace; the high-temperature furnace for the vaporization and/or oxidation of the sample at a high temperature to form water vapor and carbon dioxide gas; the analysis unit for determining the carbon content of the sample on the basis of the carbon dioxide gas produced during the oxidation of the sample, wherein the carrier gas transports carbon dioxide gas produced during the vaporization and/or oxidation of the sample to the analysis unit; and a data processing unit, which is configured to carry out the steps of the method according to one of the preceding claims; such as, the data processing unit is configured to carry out the steps of: controlling the shut-off device, controlling and/or regulating the injection unit, and determining the carbon content of the sample.

One embodiment provides that the shut-off device be configured as a valve, such as a 3/2-way valve.

One embodiment provides that the analyzer comprise: a condensation unit for condensing the water vapor produced during vaporization and/or during oxidation of the sample to form a condensate.

One embodiment provides that the analyzer comprise a humidification unit for humidifying the carrier gas by means of the condensate.

One embodiment provides that the analyzer comprise a pump for transporting the condensate from the condensation unit to the humidification unit.

One embodiment provides that the analyzer comprise a cooling unit for cooling the condensation unit, wherein the condensation unit is configured to be coolable.

One embodiment provides that the analyzer comprise a processing unit for removing carbon dioxide gas from the carrier gas before the oxidation of the sample, wherein the processing unit has a binder, such as a comprising soda lime, for binding the carbon dioxide gas from the carrier gas.

One embodiment provides that the carrier gas be ambient air, compressed air, nitrogen, or a gas mixture, such as a gas mixture composed of nitrogen and oxygen.

One embodiment provides that the analyzer comprise a filter which is arranged between the high-temperature furnace and the analysis unit and is configured for filtering acidic gases, dust, and/or aerosols.

### DETAILED DESCRIPTION

This is explained in more detail with reference to the following figures.

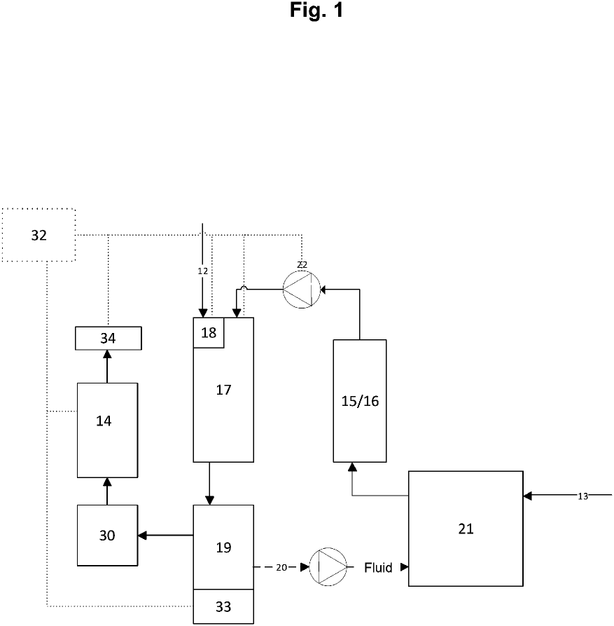

FIG. 1 shows a schematic embodiment of the claimed TOC analyzer.

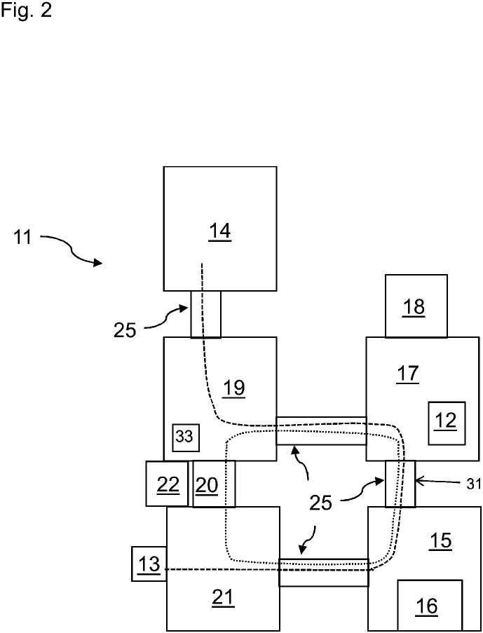

FIG. 2 shows a schematic drawing of the claimed TOC analyzer in one embodiment.

In the figures, the same features are labeled with the same reference signs.

### DETAILED DESCRIPTION

The claimed TOC analyzer in its entirety has the reference sign 11 and is schematically illustrated in FIG. 1.

The TOC analyzer 11 serves to determine a carbon content of a sample. Depending upon the type and composition of the sample, it must still be prepared for the TOC analysis (however, the sample preparation per se is not an essential part of the present application). The sample 12 is introduced, e.g., injected, into a high-temperature furnace 17 by means of an injection unit 18. The high-temperature furnace 17 is at its reaction temperature between 670 and 1,200° C., so that vaporization and/or oxidation of the sample 12 takes place. In some cases, the reaction runs by means of a catalyst. The water vapor formed is condensed in a condensation unit 19; in one embodiment, this is coolable (cooling unit 33). The water vapor can be collected in a receptacle. An expansion chamber for preventing flow of condensed liquid back into the furnace 17 can be arranged between the furnace 17 and the receptacle.

The carbon dioxide gas produced during the vaporization and/or oxidation of the sample 12 is transported using a carrier gas to the analysis unit 14, in which the carbon content is determined. The carrier gas can, for example, be ambient air, compressed air, nitrogen, or a gas mixture, in particular a gas mixture composed of nitrogen and oxygen. If the carrier gas has at least traces of carbon dioxide gas, they must be removed from the carrier gas before it is introduced into the high-temperature furnace 17 (see in this regard FIG. 2). The carrier gas is introduced into the TOC analyzer via an inlet 13. This generally takes place by means of a compressor or by means of compressed air. Frequently, regulatable pumps are also used, which are arranged in the TOC analyzer 11. The pumps are regulated such that the desired carrier gas flow is achieved, such as, for example, via a mass flow measurement. The carrier gas is typically guided through the TOC analyzer from the inlet 13 to the analysis unit 14 by means of a suitable pressure. In the flow profile of the carrier gas upstream of the analysis unit 14, a filter is arranged which is configured for filtering acidic gases, dust, and/or aerosols. The path of the carrier gas is represented by dashed lines in FIG. 1.

Between inlet 13 and high-temperature furnace 17, there is a valve 31 for stopping and starting the flow of the carrier gas through the high-temperature furnace 17. The valve 31 is, for example, a shut-off or 3/2-way valve. A 3/2-way valve is preferred here because a pressure would build up in front of a shut-off valve that would be unpleasantly dissipated by the apparatus during later opening.

More generally, the flow of the carrier gas through the furnace 17 is started or stopped via a disconnection device 31. The valve is a first embodiment. A second embodiment comprises, as a shut-off device, one or more pumps which transport the carrier gas and are then switched off. After the entire sample 12 has been vaporized, the pumps are switched back on again. The pumps are controlled, and the power can thus be set between 0 and 100%.

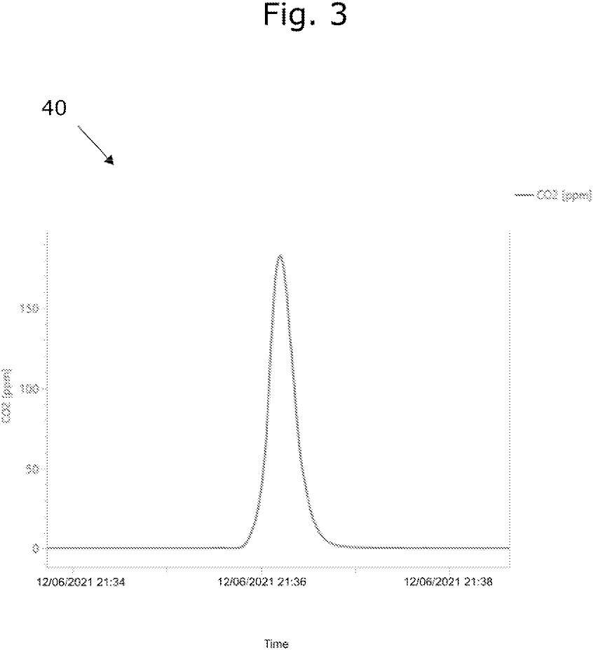

A data processing unit 32 is also shown, which is configured to control the shut-off device 31, to control and regulate the injection unit 18, and to determine the carbon content of the sample 12 via the measurement data of the analysis unit 14. This is shown in FIG. 1 by dotted lines. The analysis unit 14 comprises a non-dispersive infrared sensor (NDIR sensor, i.e., an NDIR CO₂ detector). For the determination of the carbon content, the mass flow is measured by means of a mass flow measurement 34 of the carrier gas through the analysis unit 14. Finally, measured flow is multiplied by the carbon content of the sample, wherein this product is integrated over time, and the TOC concentration of the sample is determined from the integral. FIG. 3 shows such a time-concentration diagram 40.

As mentioned, a shut-off valve which serves to shut off the carrier gas is arranged in the first embodiment directly upstream of the furnace 17 in the carrier gas flow. A second embodiment comprises a regulated pump as described above. In both cases, the carrier gas is switched off, immediately before the sample 12 is metered into the furnace 17. After the end of the metering and after the sample 12 is completely vaporized in the furnace 17, the carrier gas is switched on again.

The carrier gases is thus shut off by means of the shut-off device 31 before the sample is metered into the reactor. Then, the sample 12 is metered in slowly or in pulse-like shocks. It is maintained until all of the sample 12 vaporizes. The carrier gas flow through the reactor 17 is then restarted by opening the valve (first embodiment) or starting the pumps (second embodiment). Finally, the TOC content is calculated as described above.

In FIG. 2, the TOC analyzer 11 is shown schematically in one embodiment. The path of the carrier gas is represented by dashed lines in FIG. 2. The dotted lines approximately represent which units the water or the water vapor moves between.

In FIG. 2, the sample 12 is in the furnace 17; FIG. 1 shows the sample 12 before the injection.

As mentioned, traces of carbon dioxide gas must be removed from the carrier gas before it is introduced into the high-temperature furnace 17. For this purpose, the TOC analyzer 11 in one embodiment comprises a processing unit 15.

A binder 16, e.g., soda lime, is provided in the processing unit 15, which binder extracts the carbon dioxide gas from the carrier gas and binds it. The condensate formed in the condensation unit 19 is collected and discharged via an outlet 20 to a humidification unit 21. The outlet 20 can be configured, for example, as a valve or a siphon in order to prevent the transfer of carrier gas from the humidification unit 21 into the condensation unit 19. Optionally, a pump 22 may also be used to pump the condensate out of the condensation unit 19 and into the humidification unit 21.

The condensate is provided in the humidification unit 21 and brought into contact with the carrier gas so that the carrier gas is humidified by the condensate. When the carrier gas subsequently flows into the processing unit 15, the water vapor absorbed by the carrier gas in the humidification unit 21 can humidify the binder 16. The humidification of the binder 16 is thus ensured by an internal process of the TOC analyzer 11. The connecting pieces 25 between the various units, e.g., the connection between the humidification unit 21 and the processing unit 15, are shown in FIG. 2 by way of example as pipes, and in FIG. 1 as arrows. There is no limitation on the connections and transitions between the individual units, as well as the exact arrangement thereof.

What is disclosed and claimed is thus a TOC analyzer 11 and a corresponding and method in order to be able to use large sample volumes in catalytic high-temperature combustion and nevertheless to receive CO₂ time curves that can be integrated well. For this purpose, the carrier gas is switched off immediately before the sample metering into the furnace 17 (flow = 0 mL/min). The vaporization and the oxidation reaction thus proceed. The reaction products remain shortly behind in the reactor or on the flow side. Some seconds after metering, the carrier gas stream is switched on again, and the reaction product is flushed into the analysis unit 14. The CO₂ values thus measured are multiplied by the temporally-assigned carrier gas flow speeds, and these products are integrated. The integrals thus obtained are proportional to the TOC concentrations of the sample 12.

1. A method for determining a carbon content of a sample in a TOC analyzer, comprising the steps of:
directing a carrier gas from an inlet via a high temperature furnace to an analysis unit;
stopping the flow of the carrier gas through the high temperature furnace;
injecting the sample into the high-temperature furnace, which is used for vaporizing and/or oxidizing the sample at a high temperature to form water vapor and carbon dioxide gas;
waiting until the sample injected into the high-temperature furnace is vaporized;
starting the flow of the carrier gas through the high-temperature furnace and thereby transporting the carbon dioxide gas produced during the vaporization and/or oxidation of the sample to an analysis unit; and
determining the carbon content of the sample by means of the analysis unit on the basis of the carbon dioxide gas produced during the oxidation of the sample.

2. The method according to claim 1,
wherein the injection is performed in a pulse-like manner.

3. The method according to claim 1,
wherein the determination of the carbon content is performed cyclically.

4. The method according to claim 1,
wherein the flow is measured using a flow meter.

5. The method according to claim 4,
wherein the measured flow is multiplied by the carbon content of the sample, wherein this product is integrated over time, and the TOC concentration of the sample is determined from the integral.

6. A TOC analyzer for determining a carbon content of a sample, comprising
an inlet for a carrier gas, wherein the inlet leads via a shut-off device to a high-temperature furnace, wherein the carrier gas is used for transporting a carbon dioxide gas produced in the high-temperature furnace during an oxidation of the sample to an analysis unit;
the shut-off device for stopping and starting flow of the carrier gas through the high temperature furnace;
an injection unit for injecting the sample into the high-temperature furnace;
the high-temperature furnace for vaporizing and/or oxidizing the sample at a high temperature to form water vapor and carbon dioxide gas;
the analysis unit for determining the carbon content of the sample on the basis of the carbon dioxide gas produced during the oxidation of the sample, wherein the carrier gas transports the carbon dioxide gas produced during the vaporization and/or oxidation of the sample to the analysis unit; and
a data processing unit configured to carry out the following steps:
controlling the shut-off device,
controlling and/or regulating the injection unit, and
determining the carbon content of the sample.

7. The TOC analyzer according to claim 6,
wherein the shut-off device is configured as a valve.

8. The TOC analyzer according to claim 6, comprising
a condensation unit for condensing the water vapor produced during the vaporization and/or during the oxidation of the sample to form a condensate.

9. The TOC analyzer according to claim 6, comprising
a humidification unit for humidifying the carrier gas by means of the condensate.

10. The TOC analyzer according to claim 9, comprising
a pump for transporting the condensate from the condensation unit to the humidification unit.

11. The TOC analyzer according to claim 8, comprising
a cooling unit for cooling the condensation unit, wherein the condensation unit is configured to be coolable.

12. The TOC analyzer according to claim 6, comprising
a processing unit for removing carbon dioxide gas from the carrier gas before the oxidation of the sample, wherein the processing unit has a binder for binding the carbon dioxide gas from the carrier gas.

13. The TOC analyzer according to claim 6,
wherein the carrier gas is ambient air, compressed air, nitrogen, or a gas mixture, in particular a gas mixture composed of nitrogen and oxygen.

14. The TOC analyzer according to claim 6, comprising
a filter between the high-temperature furnace and the analysis unit for filtering acidic gases, dust, and/or aerosols.
